(19) United States
(12) Patent Application Publication
(10) Pub. No.: US 2010/0068825 A1
(43) Pub. Date: Mar. 18, 2010

(54) Method and Device for Detecting at Least One Property of at Least One Object with a Microchip

(75) Inventors: Frank Breitling, Heidelberg (DE); Michael Hausmann, Ludwigshafen (DE); Kai Konig, Walldorf (DE); Volker Linderstruth, Schriesheim (DE); Alexander Nesterov-Muller, Edingen (DE); Gloria Maria Torralba Collados, Heidelberg (DE); Volker Stadler, Heidelberg (DE); Yipin Zhang, Munchen (DE); Ralf Bischoff, Heidelberg (DE)

Correspondence Address: Saul Ewing LLP (Philadelphia), Attn: Patent Docket Clerk, 2 North Second St., Harrisburg, PA, 17101, US

(73) Assignee: Ruprecht Karls Universitat Heidelberg, Heidelberg (DE)

(21) Appl. No.: 12/516,958

(22) PCT Filed: Nov. 30, 2007
(86) PCT No.: PCT/EP07/63110

§ 371 Date: May 29, 2009

(30) Foreign Application Priority Data
Nov. 30, 2006 (DE) 10 2006 056 949.0

(51) Int. Cl. G01N 33/543; G01N 21/00

(52) U.S. Cl. 436/518; 422/82.05

(57) ABSTRACT

The present invention relates to a method and a device for the detection of at least one property of at least one object. The detection is effected by means of a microchip. The microchip has at least one readable detection pixel. In order to reduce the technical equipment outlay during object detection, the method according to the invention is characterized by the fact that the at least one object is arranged at the microchip in a spatially predetermineable position. The at least one object is exposed to illumination light in order to detect the illumination light that interacts with the at least one object or the light that is induced by the illumination light and emerges from the at least one object by means of the at least one readable detection pixel of the microchip.

The present invention relates to a method and a device for the detection of at least one property of at least one object. The detection is effected by means of a microchip. The microchip has at least one readable detection pixel.

For the purposes of the present invention, a microchip is understood to mean, in particular, a microchip comprising integrated electronic circuits for control and read-out. Such a microchip could be based on CMOS technology, for example.

In biology, chemistry, pharmacy and medicine, the term microchip or chip has come to denote usually a carrier to which arrays of reactive molecules are applied in high density and are used for screening molecular objects or test substances. Hereinafter, therefore, the term chip is used if such a carrier known from the prior art is meant.

Molecular screening by means of DNA, RNA, PNA, peptides or proteins, in particular also of mixtures of these substances, have become indispensable techniques in biological and biomedical research and in medical diagnosis. In this way different DNA and RNA sequences can be applied (“spotted”) in a high density onto a suitable carrier and coupling reactions at specific partners can be detected by means of automated readers. The following may be mentioned as an example: genome arrays for characterizing unknown genomes, cDNA arrays, gene expression arrays or oligonucleotide arrays for searching for single nucleotide polymorphisms (SNPs). In contrast to oligonucleotides, the spot or synthesis density in the case of peptide arrays is significantly lower for technical reasons. Peptide, or more precisely oligopeptide, arrays can be used for characterizing antibodies or antibody mixtures, such as e.g. blood serum, in order to search for pharmaceutically active molecules that block viral infections, for example, or modulate the function of a protein as a result of the specific binding to the latter.

In all these chip technologies for molecular screening, in particular in biology, biotechnology, chemistry, pharmacy and medicine, it is conventional practice that after application of the (fluorescence-)marked objects or test substances and specific reaction with carrier molecules on the chip, non-specifically attached reactants are washed away. The test substances that are situated on the chips and specifically bound to carriers are then read or detected by means of a microscope or by means of a specific reader on the basis of optical microscope techniques. The peptide arrays from Jerini Biotools Affymetrix GeneChip or Abbott-Vysis Genosensor may be mentioned as examples of such technologies.

The microscope-based readers are distinguished by the following components and properties:
a separate illumination light source, for example a thermal radiator or a gas discharge lamp with an optical filter by means of which only light having a predetermineable wavelength or a predetermineable wavelength range is selected in order in this way, in a manner adapted to the excitation spectrum of the fluorescent marking substances used, to excite the latter to fluorescence emission. A laser having a suitable wavelength can also be used to excite the fluorescent marking substances;
an illumination optical unit for focusing the excitation light onto the chip surface, for example in the form of a microscope objective;
a carrier for the chip after corresponding reaction with fluorescence-marked objects or test substances;
a separate detection optical unit with corresponding optical filters for the separation of excitation light and fluorescence light and a collecting optical unit for the fluorescence light;
a photodetector, preferably a fluorescence-light-sensitive CCD camera, for converting the electromagnetic or optical signals into electrical signals (by means of the photoelectric effect);
overall high procurement and operating costs and complex handling.

A further advantage of the array or read-out technology described is that signals of two or even more different fluorescent dyes can also be analyzed simultaneously. Thus, e.g. during the comparative genome hybridization with the aid of applied DNA spots (CGH matrix analysis), two test substances can be compared with one another within one experiment, whereby reliable statements concerning the binding signals become possible even when the amount of applied DNA per individual spot fluctuates from array to array. On account of the complicated production methods, in particular of highly complex arrays, such fluctuation of the applied molecular amount per spot is certainly the rule rather than the exception, whereby noise governed by the manufacturing tolerances arises in the strength of the binding signals.

In order to obtain reliable data despite this noise, during CGH matrix analysis a complex DNA mixture (e.g. as a test substance of a tumor sample) is marked with a green fluorescent dye and compared with a closely related other complex DNA mixture (e.g. as a test substance composed of normal tissue from the same patient) that has been marked with a red fluorescent dye. Mixing the two marked DNA mixtures (test substances) produces a yellow signal whenever identical amounts of DNA are present in the tumor sample and in the normal tissue sample, which compete with one another to bind to a complementary genomic DNA sequence applied on the carrier. However, if specific genome regions of the tumor sample are absent or have been duplicated, then a readily detectable red or green signal arises. Besides the color of the fluorescence signal (red, yellow or green), in this case all that is important is that the corresponding genome regions are covered by the complementary DNA sequences supplied on the array, but not the intensities of the individual signals themselves, which can fluctuate greatly owing to the dictates of manufacturing.

The principle described here for the comparative genome hybridization can, of course, also be used for analogous questions, e.g. by marking the respective antibody serum of a human before and after an immunization with red and green fluorescent dyes (e.g. by means of the Xenon Labelling Kit available from the company Molecular Probes). If a peptide array is dyed with a mixture of these two antibody sera, then peptide spots dyed red and green would reveal differences in the antibodies present before and after the immunization, these differences being of great interest for the success of inoculation, wherein the signals obtained—as described for the CGH —would be largely independent of manufacturing-dictated fluctuations in the applied amount of the individual peptides. This principle of the comparative binding of test substances to a molecular library can be applied to all of the test substances or objects described above (e.g. virus, bacteria and cell variants, defective/functional extracellular matrix, protein mixtures of closely related cells, allergens, molecules obtained from arthritic/healthy kneecaps, etc.), but in particular to closely related mixtures of these test substances. In this case, the company Molecular Probes, in particular, offers a large number of fluorescent dyes which can be coupled very easily to amino groups, OH groups and sugar molecules, such that almost all biological objects or test substances can be converted with fluorescent dyes.

One disadvantage of these chip technologies and read-out methods consists in the relatively high technical equipment outlay for the external detection of the fluorescence signals on the chip. Owing to geometrical and optical optimization of these readers, the conventional chips are fixed in form, size and occupation density. Variations in the chip design require a complete adaptation of the detection unit.

Therefore, the present invention is based on the object of specifying and developing a method and a device of the type mentioned in the introduction by means of which the technical equipment outlay can be reduced and, in particular, a miniaturization of the array technology becomes possible.

The method according to the invention of the type mentioned in the introduction achieves the above object by means of the features of patent claim 1. Accordingly, such a method is characterized by the fact that the at least one object is specifically bound to or arranged at the microchip in a spatially predetermineable position. The at least one object is exposed to illumination light in order to detect the illumination light that interacts with the at least one object or the light that is induced by the illumination light and emerges from the at least one object by means of the at least one readable detection pixel of the microchip.

Since the object or objects is or are specifically bound to or arranged at the microchip in a spatially predetermineable position or in a spatially defined position, the method according to the invention enables a pixel-correlated detection of the objects. This is because, in particular, a detection pixel generally detects the light from the object which is spatially at the least distance from the detection pixel. For the purposes of the present invention, a detection pixel should be understood to mean, in particular, a detection region of the microchip which is arranged at a predetermineable distance from the microchip surface or at a predetermineable depth of the microchip and by means of which light can be detected.

Consequently, detection pixels are optical sensors, such as photogates or photodiodes, for example, which generate an electrical signal. Their signals can be amplified and/or measured in temporally resolved fashion on the same microchip during the read-out process. The signals can be measured or detected by digital-to-analog converters or by programmable analog thresholds and discriminators. Consequently, the detection pixels have electrical charges or potentials which could be read out individually. Therefore, unlike in the case of a CCD camera, it is not necessary to detect all the pixels (e.g. pixel shift) simultaneously as accurately as possible. The read-out and possibly also the amplification of the optical signals can therefore also be effected line by line for example by means of a series of illumination events. In general, it is preferred for the detection pixels to be read in groups.

In principle, it is also conceivable for the object to be exposed to an electromagnetic wave instead of being exposed to illumination light. Accordingly, the electromagnetic wave that has interacted with the object is then detected by means of a detection pixel. It is furthermore conceivable for a further electromagnetic wave that is induced by the electromagnetic wave and emerges from the at least one object to be detected by means of the at least one readable detection pixel. This will be discussed in more detail below.

Suitable objects or test substances include all kinds of molecules which are relevant in biology, chemistry, pharmacy and medicine and which, in particular, can specifically bind to connection or linking objects that are arranged immobile on the microchip. These specific bindings play an important part in determining the molecular properties of the objects or test substances, but also of the connection objects bound on the microchip, which are embodied in the form of oligomers. Typical examples of such test substances are antibodies, proteins, peptides, enzymes, DNA molecules, RNA molecules, synthetic pharmaceuticals and mixtures of these substances.

It has been recognized according to the invention that the microchip does not just serve as a carrier for the object or objects to be detected and/or for the suitable connection objects or linking objects. Rather, the microchip also serves for detecting the light which has interacted with the object or objects or which has been induced by the illumination light at the object. If one assumes—as described in the introduction—a very compact packing density of the objects to be detected on the microchip, the object detection of the densely arranged objects can be effected simultaneously by means of the microchip acting as a carrier, to be precise without providing further optical components such as detection optical units and CCD cameras for this purpose. For this purpose, the microchip or object carrier does not have to be moved relative to a detection optical unit (for example by means of a microscope stage) in order to be read completely. The detection of the light coming from the object takes place where it arises rather than at a distance customary in microscopy, where the light to be detected has to pass through a multiplicity of optical components in order finally to be detected e.g. by a CCD chip of a CCD camera with a comparatively low quantum efficiency. In this respect, it is also not absolutely necessary for—in comparison with detection by means of a CCD camera—a plurality of images or detection cycles to be recorded and averaged, since losses of the detection light as a result of partial reflections at optical components such as lenses, etc. do not occur in the method according to the invention.

Consequently, unlike in most array technologies, the microchip for the purposes of the present invention serves only as a “passive” carrier which only has the task of anchoring the applied connection/linking objects, molecules or objects on an essentially two-dimensional surface.

In the exemplary embodiments, in particular, which will be discussed below, it is clarified that the method according to the invention can be carried out and optimized by means of a suitable combination of illumination optical unit, choice of the absorption or fluorescent marking substances for the objects and construction or arrangement of the photodetectors with regard to absorbent silicon layer thicknesses, surface layers and/or dopings, and the abovementioned read-out components such as detection optical units and separate photodetector can then be obviated. It is thereby possible advantageously to detect a large number of different objects, presupposing a corresponding marking and (predetermined or known) object positioning on the microchip, in a relatively short time and thus to record their information content.

Connection/linking objects and/or the object or objects can be arranged or applied on the microchip in a predetermineable or defined position in various ways. Preferred method steps by which the connection/linking objects and/or the objects can be applied on the microchip are discussed below.

Thus, the connection/linking objects and/or the objects could be positioned on the microchip and bound there by means of electrostatic attraction forces. For this purpose, the microchip has at least one electrode pixel. An electrode pixel could be embodied in the form of a high-voltage pixel or a high-voltage electrode. A voltage lying in a range of 30 to 100 V can be applied to an electrode pixel. The voltage can be positive or negative. Such a micropixel usually has an edge length of approximately 30-100 μm. The microchip can thus have approximately 10 000 to 100 000 pixels/cm². Consequently, in this embodiment variant, it is possible to take as a basis a microchip which implements an array of electrode pixels whose electrical potentials can be freely programmed, i.e. activated or deactivated, individually or in groups. The programming of such a microchip is carried out via customary computer interfaces such as I²C, USB or the like, wherein the microchip implements the necessary control and status registers that are orchestrated or driven by suitable software of a host or control computer (e.g. of a PC).

It is then possible for the connection/linking objects or the at least one object to be attached to the microchip in such a way that an electric field is generated selectively by means of the at least electrode pixel. Said electric field is generally active in a spatially delimited manner for the objects. By means of the electric field, at least one object or at least one connection/linking object is attached to a surface region of the microchip which is assigned to the electrode pixel. The precondition for this is that the object or the connection/linking object is electrically charged. This will be discussed in specific detail in the exemplary embodiments. Attachment should be understood to mean, in particular, a specific arrangement of an object on the microchip.

Particularly preferably, the selective attachment of connection objects is repeated. A plurality of connection/linking objects are thereby synthesized pixel-by-pixel to form a respective (if appropriate more complex and/or specific) connection object. The connection/linking objects synthesized in this way serve for the specific attachment of the objects to be detected. Synthesizing can be understood to mean, in particular, the protective group-based combinatorial (solid-phase) synthesis of linear oligomers from amino acids (Merrifield synthesis) to form peptides or nucleotides to form RNA (ribonucleic acid) or DNA (deoxyribonucleic acid) oligomers, which is known from the prior art, see for example Merrifield R. B. and Stewart J. M., 1965, Automated peptide synthesis, Nature 207, 522-523. The same chemistry can also be used to synthesize PNA (peptide nucleic acid) oligomers combinatorially in a given sequence of nucleic acids which chemically have commonalities with RNA and DNA but a peptide backbone (e.g. N-(2-aminoethyl)glycin). Oligonucleotides or oligoribonucleotides can also be synthesized in the same way.

This manner of application is comparable with the method for applying substances to a carrier that is described in EP 1 140 977 B1, which describes a method for applying substances to a carrier, in particular monomers, for the combinatorial synthesis of molecular libraries, which method is suitable for the combinatorial synthesis of a multiplicity of different molecules on the electrode pixels of a microchip configured in the form of a CMOS chip. Suitable molecules include, for example, peptide oligomers, DNA oligomers, RNA oligomers or PNA oligomers (called oligomers for short hereinafter). Said substances comprise amino acid or nucleotide monomers in a solid state of matter. These monomer carriers can be produced in the form of microparticles having a typical diameter of 10 μm and serve as a transport unit for the monomers onto the electrode pixels of the microchip. The dissertation by Alexander Nesterov-Müller, Faculty of Physics and Astronomy at The University of Heidelberg, 2006, describes methods by which the microparticles can be electrically charged and applied to the electrode pixels of a microchip selectively, in a positionally accurate manner. In this case, it is advantageous to apply high voltages of the order of magnitude of 30-100 V selectively to the electrode pixels. Owing to the very small dimensions of the structures on the CMOS microchips, at said voltages it is possible to attain very high field strengths, which can virtually attain the breakdown voltage in air. This in turn is very advantageous for the positionally accurate positioning of the charged microparticles with the aid of the voltages applied to the individual pixels.

EP 1 140 977 B1 likewise describes a method for the synthesis of a carrier-bound array of oligomers. For this purpose, the abovementioned microparticles are applied to the carrier layer by layer and subsequently melted. The monomers are thereby mobilized, with the result that they can couple to the carrier. Afterward, non-bound substances are washed away and the non-permanent protective group such as e.g. Fmoc (in the case of peptide or PNA synthesis) or trityl (in the case of oligonucleotide synthesis) is detached. By cyclically repeating the method, e.g. analogously to the standard Merrifield synthesis, a plurality of layers of particles are applied and the oligomers and the connection objects are synthesized.

If connection objects are attached in a positionally accurate manner or in a predetermined position on the microchip in at least one method step, at least one object can be specifically attached to the at least one connection object. Such an object could comprise for example an antibody or an antibody mixture, proteins, peptides, DNA molecules, RNA molecules, PNA molecules, sugar molecules or bacterial lipopolysaccharide.

The test substances or objects to be examined can be specifically linked to the multiplicity of different connection objects (embodied e.g. in the form of oligomers) and to the microchip by the method steps described above, by means of the chip surface being brought into close contact with the test substances/objects in suitable incubation media such as aqueous buffers or alternatively, if appropriate, with the aid of a gas phase. Through suitable configuration and combinatorial formation of the connection objects on the microchip, unknown test substances/objects or substance mixtures can be systematically analyzed according to specific molecular properties (molecular screening). However, it is also possible to use in particular known test substances therefor in order to find one or a plurality of connection objects which bind thereto and which then prevent e.g. a virus particle from entering into its host cell. The following may be mentioned as some application examples for molecular screening: genome screening or mRNA screening of unknown nucleotide sequences, DNA sequencing with the aid of oligonucleotide arrays, antibody screening in blood sera, e.g. in the case of viral infections, characterization of enzyme substrates, in particular of kinases or phosphatases, examinations regarding protein-peptide bonds, examinations regarding molecular interactions of therapeutic pharmaceuticals with cell surfaces and peptide or protein targets. Accordingly, the connection objects are then embodied in such a way that the objects to be examined can in each case bind specifically to them.

Furthermore, it is also conceivable for the microchip or the electrode arrays to be chemically occupied by functional groups, with the result that amino acids or nucleotides can be bound on the electrodes. This is known for example, from the publication by Beyer et al., Biomaterials, 27, 3505-3514, 2005, or the dissertation by Mr. Mario Beyer, Faculty of Chemistry, University of Heidelberg, 2005. It is likewise possible to apply and/or bind at least one specific and/or synthesized connection object to the functional groups of the microchip surface. In this case, the connection objects are likewise embodied in such a way that the objects to be examined can in each case bind specifically to them. In accordance with this embodiment, therefore, the connection objects are not synthesized on the microchip. This is done beforehand in a different manner. The objects and/or connection objects could then be applied on the microchip by means of the spotting method and/or, the micromanipulation method in the form of positionally accurately deposited microdroplets and be fixed with the microchip by a chemical coupling by means of corresponding functional groups. These application methods are known from the prior art, and so they are not discussed any further here.

A linking/connection object can comprise a reactive molecule, in particular an oligomer, a peptide oligomer, a DNA oligomer or a PNA oligomer of defined amino acid or nucleotide sequence.

Furthermore, it is conceivable for the at least one object to be positioned on the microchip by applying a film. In this case, the at least one object is positioned on the film in a spatially predetermineable manner. This positioning of the at least one object on the film could be applied or fixed thereto by means of the method described above. In an alternative embodiment, the at least one object could be at least partly surrounded by a medium, for example a gel. In this case, the medium is embodied in such a way that the relative position of the objects—in particular among one another and/or with respect to the microchip or with respect to the individual detection pixels of the microchip—remains essentially unchanged thereby. The medium together with the objects can then be applied to the microchip.

The at least one object is illuminated with illumination light having at least one predetermineable wavelength or a predetermineable wavelength range. The illumination can be effected in punctiform or areal fashion. A punctiform illumination of individual objects or of a plurality of objects can be effected by means of a focused light beam. The entire microchip could also be illuminated e.g. by means of a collimated light beam. The wavelength range of the illumination light can extend from 280 nm to 1000 nm, in particular from the UV-C to the near IR.

If luminescence light emerging from the object is intended to be detected or verified, it is desirable that no illumination light that serves for exciting the luminescence reaches the detection pixels. Only the luminescence light is intended to be detected by the detection pixels. The microchip is generally embodied in such a way that its detection pixels are arranged at a distance from the microchip surface, for example at a depth of approximately 500 to 1000 nm. Accordingly, provision is preferably made for illuminating the objects in such a way that the illumination or excitation light does not penetrate, or penetrates only slightly, into the microchip. This could be realized on the one hand by illumination by means of suitable optical components. On the other hand, by means of a suitable choice of the wavelength or the wavelength range of the illumination light, the penetration depth thereof could be kept small, e.g. of the order of magnitude of 100 nm. In principle, in the case of silicon chips, the penetration depth is smaller in the case of light having a short wavelength than in the case of light having a longer wavelength. Therefore, with short-wave UV light, essentially only the microchip surface and the luminescence-marked test substances/objects could be illuminated, but not the detection pixels. However, on account of the Stokes shift, the luminescence light emitted by the object has a longer wavelength and therefore has a larger penetration depth into the microchip and can therefore reach a photosensitive layer or the detection pixels in the microchip. By means of the photoelectric effect, an electrical signal (e.g. in the form of a photocurrent) or a charge transfer can be initiated in the detection pixel. By means of electronic circuits integrated in the chip, these signals can be amplified, digitally conditioned and made available for read-out by an external computer, such that the individual objects can be detected. In an advantageous manner, neither microscopic read-out systems nor image recognition systems are necessary for this purpose.

In accordance with one preferred embodiment, the at least one object could be illuminated evanescently. This could be effected with the aid of a prism, for example, wherein the prism is arranged at a predetermineable distance relative to the microchip or to the objects. Illumination light could then be coupled into the plasma in such a way that, on account of the total reflection that takes place in the prism, an evanescent field forms outside the prism and on the side facing the microchip or the objects. The at least one object is illuminated by means of this evanescent light field. The intensity of the evanescent light field decreases exponentially with the distance from the interface of the prism and, given a suitable arrangement of the prism relative to the microchip, detectable intensities of the illumination light typically penetrate less than 100 nm into the microchip from the surface thereof. As a result of this, the illumination light does not reach the depth at which the detection pixels of the microchip are situated. Accordingly, by means of such object illumination, the illumination light is not detected by the detection pixels. By contrast, for example the fluorescence light emerging from the object can be detected by the detection pixels since the fluorescence light emerging from the objects has a longer wavelength and/or penetrates deeper into the microchip.

The illumination light is generated by means of a light source that emits continuous and/or pulsed light. The light source could comprise a laser, a thermal radiator or a gas discharge lamp. In general, provision will be made of an illumination optical unit and further optical components (e.g., mirrors), if appropriate, by means of which the light emitted by the light source can be directed or focused onto the microchip.

In accordance with one preferred embodiment, the object is illuminated with pulsed light. The at least one detection pixel is read in an illumination pause. Although the illumination light reaches the detection pixels during the illumination phase if its penetration depth is high enough, the signal possibly detected during the illumination phase is not taken into account. In the illumination pause, however, no illumination light reaches the detection pixels, such that for example only the luminescence light induced by the illumination light (presupposing a sufficient lifetime of the luminescent dye) can then be detected by the detection pixels.

In specific terms, luminescent or fluorescent marking substances having a luminescence or fluorescence lifetime of the order of magnitude of milliseconds could be used for object marking. As a result of switching off the detection pixels or as a result of not taking account of the signals generated by the detection pixels during the illumination and subsequently switching on the detection pixels during the time-delayed fluorescence emission, only the fluorescence light is detected. It is thus possible to differentiate the illumination light from the fluorescence light.

The temporal synchronization with the illumination system can be carried out with the aid of reference photosensors or reference detection pixels by means of suitable pulse sequences before or during the measurement, with the result that there is no need for an external synchronization infrastructure between the illumination system and the microchip. Said reference photosensors can also be used to detect and electronically correct temporal alternations of the illumination system. The signals of the reference photosensors can also be used for calibration purposes—for example of the illumination intensity distribution at the microchip surface.

In a further embodiment, the object is marked with an absorption dye. That proportion of the illumination light which passes through the object and through the absorption dye to the respective detection pixel is detected. In this respect, after marking with the object, the absorption dye can be regarded as being associated with the object, such that an interaction of the object with the illumination light within the meaning of claim 1 can also be understood as an interaction with the absorption dye. In this case, it is expedient to choose the illumination light with regard to its spectral property in such a way that it is absorbed by the absorption dye and that it has a large penetration depth into the microchip in order that an electrical signal (e.g. in the form of a photocurrent) is initiated in those detection pixels which are not covered with absorption-marked objects. The detection pixels which are covered with absorption-marked objects will initiate no or only a negligible signal.

A specific absorption detection could also be effected in such a way that there is applied on the microchip a layer which changes its optical properties—e.g. changes color—when it comes into contact with the corresponding reactant. Such a reactant can be specifically spatially attached to the surface of the microchip in each case with the aid of the electrode pixels. Such a layer could comprise for example palladium-tungsten, in specific terms Pd—WO₃. Such a layer turns blue upon contact with molecular hydrogen, whereby the detection of potentially catalytic events by means of the detection pixels of the microchip is possible since the absorption properties of the layer have changed locally as a result of the color change. The dissociative adsorption of hydrogen leads to the reduction of WO₃ to form tungsten bronzes. The latter are composed of tungsten oxides having different valences (+5, +6) which have a deep blue coloration and the conductivity of which rises by a factor of more than 106 upon contact with 1% H₂. It is therefore possible to use such layers or films in combination with the detection pixels as optical sensors, wherein the detection sensitivities can be up to 3 ppm. On the basis of the conductivity of tungsten bronzes it is already possible to realize resistive hydrogen detection with the aid of semiconductor structures. Pd—WO₃ layers can be produced either by means of sol-gel methods, by thermal vapor deposition or by means of sputtering. In this respect, in accordance with this embodiment variant, absorption detection of absorption dye specifically bound to objects is not effected, rather the absorption properties of those regions of the layer of the microchip which have changes with regard to their optical properties on account of specifically attached objects or reactants are detected. As a result of this, these regions absorb a larger part of the illumination light than regions of the layer which have remained optically unchanged.

In principle, it is also conceivable to configure the layer of the microchip with a high absorption coefficient and, after specific reaction with a corresponding reactant has taken place, the optical properties of the changed regions change toward a lower absorption coefficient.

Particularly preferably, it is provided that the object is specifically marked with at least one luminescent dye, and/or that the object is specifically marked with at least one nanocrystal, capable of luminescence. The at least one luminescent dye is excited to luminescence by the illumination light. The luminescence light is detected by a detection pixel. The luminescent dye could comprise a fluorescent dye or a phosphorescent dye. The nanocrystal could be capable of fluorescence or luminescence. In principle, all illumination and detection variants which are customary in fluorescence microscopy can also be applied to the method according to the invention, wherein the special characteristics should be taken into account, such as e.g. the fact that there are usually no separation filters provided in the microchip.

Thus, the objects can be marked with light-absorbing molecules and/or fluorescent molecules and/or fluorescent semiconductor nanocrystals directly or indirectly, that is to say via secondary linker molecules such as e.g. second antibodies. As an alternative the objects or test substances can displace previously bound, in particular marked substances or modulate the optical properties thereof. Examples of this include the cleavage of a fluorescence-marked peptide portion by the enzymatic activity of a protease, or the displacement of a fluorescence-marked antibody by a competitive binding of the test substance, wherein the antibody can be specifically bound to the individual oligomer, or alternatively binds to an always identical fusion portion of the different oligomers, wherein this binding competes with the binding of the test substance to the variable part of the oligomer. This last-mentioned point would have the advantage that the test substance need not be marked separately.

The fluorescent dyes can be dyes which predominantly comprise organic dyes and which can be excited in a defined wavelength range between UV-C (ultraviolet-C) and IR (infrared). Compared with the wavelength of the illumination light, these dyes emit in a wavelength range shifted to the long-wave (Stokes shift).

A further characteristic for identifying a fluorescent dye may also be the fluorescence lifetime or fluorescence decay time of the dye molecule.

By contrast, absorption dyes absorb part of the electromagnetic spectrum and convert this energy into non-optical interactions.

Semiconductor nanocrystals, referred to for short as nanocrystals or else as quantum dots, have a typical size to 10 to 100 nm. They are usually composed of the corresponding semiconductor material (e.g. CdSe) as core and an activated and/or modified surface for binding to molecular partners. Nanocrystals are distinguished by the fact that, in contrast to organic dyes, they usually do not exhibit fading of the fluorescence. While the excitation spectrum is primarily determined by properties of the core material of the nanocrystals, the fluorescence intensity and the Stokes shift and hence the spectrum of the fluorescence emission depend not only on the properties of the material but also on the hydrodynamic radius of the nanocrystals and the direct molecular environment thereof. The hydrodynamic radius is the particle radius prior to a binding of binding molecules. The radius can additionally change as a result of the binding molecules, but this primarily does not influence the fluorescence wavelength.

Accordingly, for specific applications of the method according to the invention it can be provided that the nanocrystal has a predetermineable hydrodynamic radius, and/or that the nanocrystal has a predetermineable excitation and emission spectrum, which preferably has a large Stokes shift. In principle, nanocrystals comprise a core composed of semiconductor material. As an alternative, the nanocrystal can comprise a core composed of lanthanide material, for example a europium compound. The nanocrystal can likewise comprise a coating which promotes a—preferably specific—binding of the nanocrystal to an object.

Preferably, the fluorescent dye has a predetermineable—in particular high—Stokes shift. Such a fluorescent dye could comprise lanthanide chelate.

The fluorescent dye or the fluorescent nanocrystal preferably has a predetermineable fluorescence lifetime. The latter could preferably be greater than or equal to 1 ms. The objects are specifically marked with the fluorescent dye or the fluorescent nanocrystal. The objects can be illuminated by means of pulsed illumination light, and the detection pixels are activated and read in the illumination pauses.

Preferably, at least one detection pixel is provided which directly detects the illumination light. On the basis of the detection signal of the detection pixel it is ascertained whether an illumination pause is present. As an alternative or in addition, on the basis of the detection signal of the detection pixel—for example for calibration—the local illumination situation can be inferred, in particular the local illuminance. This has already been explained in connection with reference photosensors provided on the microchip.

In a further preferred embodiment, objects are specifically marked with at least two fluorescent dyes having different excitation properties. One of the fluorescent dyes is excited to fluorescence by means of illumination light having a first excitation wavelength for a predetermineable time interval. Afterward the other fluorescent dye is excited to fluorescence by means of illumination light having a second excitation wavelength, which is generally different from the first excitation wavelength, for a further predetermineable time interval. The fluorescence light from the two fluorescent dyes is detected temporally successively. In this case, too, the illumination light can be pulsed and the fluorescent dyes can be chosen in such a way that they have a fluorescence lifetime long enough for the fluorescence light still to be detectable by the detection pixels in the illumination pauses.

This method can be employed in the case of comparative genome hybridization. For this purpose, the different fluorescent dyes used have to be able to be excited by excitation light having different wavelengths, wherein the excitation light reaches only a small penetration depth into the chip, as described. The fluorescence light emitted in turn should be able to penetrate comparatively deep into the chip, such that it reaches the detector units and can correspondingly be detected.

An alternative to the temporally offset detection of two different fluorescent dyes can involve specifically marking the objects with at least two fluorescent dyes having different emission properties. The two fluorescent dyes can be excited by means of illumination light having a predetermined wavelength, however, on account of their excitation properties. Accordingly, the two fluorescent dyes can be simultaneously excited to fluorescence by illumination light having a predetermineable wavelength. The fluorescence light from the first fluorescent dye has an emission spectrum having a predetermineable first penetration depth into the microchip. The fluorescence light from the second fluorescent dye has a second emission spectrum having a predetermineable second penetration depth into the microchip. The two fluorescent dyes are chosen in such a way that the first penetration depth is greater than the second penetration depth. The detection region of the detection pixels is arranged at least two different distances from the microchip surfaces in the microchip, such that the fluorescence light from the first fluorescent dye is detected by the detection pixels that are at a further distance from the microchip surface and the fluorescence light from the second (and, if appropriate, of the first) fluorescent dye is detected by the detection pixel that are at a lesser distance from the microchip surface.

Although traditional optical filters or focusing elements could be integrated into the microchip, it is not necessary for them to be integrated therein. As a result, it is possible to carry out the read-out of the relevant information or the detected photons in the field with minimal outlay. The physical coupling between individual connection object and associated photodetector entails a data reduction in the detection of the individual binding events. As a result, each connection object can be assigned a (measured) photocurrent very easily, such that complicated and error-susceptible image recognition systems or the like can be dispensed with. Moreover, in this case, on account of the pixel-correlated arrangement of the objects at the microchip, a spatial assignment of the detected signals to the detected objects can largely be ensured, which, in conventional microscopic detection methods, has to be calculated by an alignment—which is complicated under certain circumstances—of the detected signals with respect to the known arrangement on the carrier.

Very generally, the at least one object could be exposed to an electromagnetic wave instead of illumination light. The electromagnetic wave that interacts with the at least one object or a further electromagnetic wave that is induced by the electromagnetic wave and emerges from the at least one object could then be detected by means of the at least one readable detection pixel of the microchip.

From a device standpoint, the object mentioned in the introduction is achieved by means of the features of claim 26. Accordingly, the device mentioned in the introduction is characterized by the fact that the at least one object is arranged and/or can be arranged at the microchip in a spatially predetermineable position, and that the at least one object can be exposed to illumination light in order to detect the illumination light that interacts with the at least one object or the light that is induced by the illumination light and emerges from the at least one object by means of the at least one readable detection pixel of the microchip.

The device according to the invention therefore advantageously combines the functioning of an object carrier, on the one hand, which—as already described above—enables a very considerable object density in a small space. On the other hand, by means of the device according to the invention, the objects arranged thereon can be detected virtually directly or their information content can be read out.

Especially preferably, the device according to the invention is provided for carrying out a method as claimed in any of claims 1 to 25. For a person skilled in the art, with knowledge of the method according to the invention as claimed in any of claims 1 to 25, carrying out said method on a device according to claim 26 is largely deducible. Therefore, reference is made to the preceding part of the description in order to avoid repetition.

In one embodiment, the microchip is based on MOS technology (Metal Oxide Semiconductor). A microchip based on CMOS technology (Complementary Metal Oxide Semiconductor) is preferably used. As an alternative, the microchip or at least part of it could be based on NMOS technology (Negative conducting channel Metal Oxide Semiconductor), and/or on PMOS technology (Positive conducting channel Metal Oxide Semiconductor). What type of microchip technology is used can depend on the concrete application and on the choice of the illumination light used, the specific markers and the other boundary conditions.

CMOS technology permits the production of highly complex microchips which can have pixel matrices situated at their surface, said pixel matrices comprising a multiplicity of high- or low-voltage electrodes having a typical edge length of 30 μm to 100 μm, for example, with the result that 10 000 to 100 000 electrode pixels/cm² can be arranged. The pixel electrodes can be individually addressed. Detection pixels can be integrated or arranged in or near to said pixel electrodes and the photosignals from the detection pixels or a pixel array comprising detection pixels can be read out individually.

Especially preferably, a detection pixel has a light-sensitive electronic unit, in particular a photodiode or a photogate. The quantum efficiency of a photogate is usually lower than that of a photodiode. Less noise is caused by the photogates since there no charges flow through an n-p junction. Photogates have an excellent temperature stability. The quantum efficiency is higher in the case of photodiodes than in the case of photogates. The quantum efficiency of photogates is extremely low in the case of illumination light having a short wavelength of less than 400 nm. Accordingly, photogates can be used for fluorescence applications, for example, if the fluorescent dye is excited by means of short-wave illumination light and the fluorescence emission takes place in the long-wave spectral range.

In accordance with one preferred embodiment, the microchip has integrated electronic circuits for driving and/or for reading the detection pixels and/or for driving the electrode pixels. The detection pixels could be able to be read individually or in groups. The programming and read-out of this chip can be carried out via customary computer interfaces such as I²C, USB or the like, wherein the necessary control and status registers can be implemented in the microchip, said register being orchestrated by suitable software of a host computer (e.g. of a PC). For this purpose, in general no particular requirements in respect of latency or throughput are imposed on this interface. The data read out can be buffer-stored on the chip in corresponding data memories and be read out by the computer at a relevant time. If necessary, the microchip could also have more extensive analysis units realized at the hardware level. One example thereof could be a digital Fourier analysis which is implemented as hardware and by means of which the autocorrelation function of a fluorescence signal can be evaluated, for example. For this purpose, it is then advantageously unnecessary to digitize the fluorescence signal with a high temporal resolution and to calculate said signal by means of a software routine, which is computer-intensive under certain circumstances. In this respect, the microchip can have both detection and evaluation units, such that the microchip ideally communicates essentially only the desired results to a control computer.

Especially preferably, the microchip has at least one electrode pixel. The electrode pixel can be embodied in the form of a high- or low-voltage pixel. A positive or a negative voltage can be applied thereto by means of a corresponding drive device, which voltage can preferably be set in a continuously variable manner.

For communicating with an external drive system, the microchip has at least one driving and/or reading interface, which, in particular, is embodied in the form of an I²C (Inter-Integrated Circuit Bus) or a USB (Universal Serial Bus) interface. In principle, a contactless reading interface is also possible, as is used in the case of transponders, for example. Consequently, the microchip can be driven and/or read by a control computer.

Furthermore, the microchip could have means for amplifying and/or conditioning the signals which can be read from a detection pixel. For this purpose, in specific terms, preamplifiers based on semiconductor technology could be provided, for example based on PMOS and/or NMOS technology.

The microchip is wetted or occupied by the objects and/or connection objects described above. These are applied to the microchip generally in solid form or in a solution. The objects and/or the connection objects can be electrically conductive. Therefore, in one preferred embodiment, for electrical insulation, the microchip is provided with a coating. Said coating could comprise silicon nitride, for example. A layer comprising at least one generic type of an—in particular organic—polymer and/or an element of the generic type of the polyethylene glycols and/or a silanization layer could be provided on the insulation coating and/or directly on the microchip. Preferably, the layer comprises a mixture of these substances. Connection objects and/or objects can be attached or chemically bound to such a layer. This binding of connection objects, in particular, is promoted by the silanization or polyethylene glycol layer.

Some exemplary embodiments of the method according to the invention are discussed below in specific terms:

In a first exemplary embodiment, reactive molecules as connection objects are synthesized or spotted by known techniques onto the electrode pixels having a given size in a pixel array of the microchip and are bound to the microchip surface by suitable chemical groups. Suitable reactive molecules or connection objects include, in particular, oligomers, e.g. peptide oligomers, DNA oligomers, PNA oligomers of defined amino acid or nucleotide sequence. Using standard methods, objects or test substances are marked with fluorescent dyes and/or semiconductor nanocrystals (quantum dots) having a specific excitation and wavelength-shifted emission spectrum and are applied to the microchip surface for specific binding to the reactive molecules/connection objects. Suitable objects include e.g. antibodies, or antibody mixtures, DNA molecules, RNA molecules, sugar molecules (for example form the extracellular matrix or bacterial lipopolysaccharide). Fluorescent dyes can be for example fluorescein (derivatives), cyanine (derivatives) or rhodamine (derivatives). Semiconductor nanocrystals (quantum dots) can be CdSe quantum dots having a defined size. After specific binding and separation of non-bound test substance (e.g. by washing) the fluorescence is excited by evanescent illumination. For this purpose, for example in one preferred embodiment, a microprism is arranged at a fixedly predetermined distance relative to the chip surface and is illuminated in total reflection. As a result of the exponential decrease in the evanescent field of the illumination within the dimension of a wavelength, detectable intensities of the excitation light typically penetrate less than 100 nm into the surface of the microchip and can thus be differentiated from the fluorescence light from the marking molecules/semiconductor quantum dots, which has a higher penetration length. By choosing an interval of the excitation spectrum in the blue or UV, this effect can additionally be reinforced on account of the low transmissivity of silicon.

In accordance with a second exemplary embodiment, reactive molecules or connection objects are synthesized or spotted by known techniques onto the electrode pixels having a given size in a pixel array and are bound to the microchip surface by suitable chemical groups. Connection objects could be, in particular, oligomers, e.g. peptide oligomers, DNA oligomers, PNA oligomers of defined amino acid or nucleotide sequence. By means of methods according to the prior art, objects or test substances, e.g. antibodies or antibody mixtures, DNA molecules, RNA molecules, sugar molecules (for example from the extracellular matrix, or bacterial lipopolysaccharide), are marked with lanthanide chelates and/or lanthanide semiconductor nanocrystals (quantum dots) having a defined size and are applied to the microchip surface for specific binding to the reactive molecules/connection objects. After specific binding and separation of non-bound test substance (e.g. by washing), the fluorescence is excited by focused far field illumination by means of illumination light in the wavelength range of less than 400 nm. In this wavelength range, the penetration depth into the microchip is less than 100 nm and can be separated from the actual detection pixel by corresponding (doped) silicon layers. The lanthanide fluorescence chelates or semiconductor quantum dots fluoresce in a wavelength range of greater than 600 nm, the light quanta of which penetrate right into the photodetector of the microchip (typically 1-3 μm) and thus initiate a photosignal at the detection pixels.

In accordance with a third exemplary embodiment, connection objects and objects in accordance with the first or the second exemplary embodiment are applied to the microchip. After specific binding and separation of non-bound test substance (e.g. by washing), the fluorescence is excited by focused far field illumination in the wavelength range of less than 400 nm by means of a pulsed light source. The fluorescence lifetime (decay time) of lanthanide dyes is of the order of magnitude of a few milliseconds. It thereby becomes possible to turn off the detection pixels or the photodetectors in the electrode pixels during the illumination given sufficiently short illumination pulses and subsequently to switch them on for the time-delayed fluorescence detection.

In accordance with a fourth exemplary embodiment, connection objects in accordance with the first or the second exemplary embodiment are applied to the microchip. This is followed by derivatizing two different, closely related test substances 1 and 2 with respectively different fluorescent molecules 1 and 2 and mixing them in equal amounts. After specific binding of the test substances to the connection objects and separation of non-bound test substances (e.g. by washing), the fluorescence 1 is excited by focused far field illumination in the wavelength range of between 300 and 400 nm by means of a pulsed light source and, during the dark phase of the excitation light, the fluorescence signals 1 are read out by means of the photocurrent 1 induced thereby in the individual detection pixels. Afterward, the fluorescence 2 is excited by focused far field illumination in the wavelength range of less than 300 nm by means of a pulsed light source and the fluorescence signals 2, or the photocurrents 2 induced thereby are read out during the dark phase of the excitation light. The quotient of the photocurrents 1 and 2 is subsequently calculated for each individual detection pixel. Said quotient is a measure of the ratio of the test substances 1 and 2 bound to the respective individual connection objects.

In accordance with a fifth exemplary embodiment, connection objects and objects in accordance with the first or the second exemplary embodiment are applied to the microchip. By means of methods according to the prior art, the objects or test substances are marked with absorption dyes and applied to the microchip surface for specific binding to the connection objects. After specific binding and separation of non-bound test substance (e.g. by washing), the microchip is illuminated by focused far field illumination in the wavelength range of greater than 400 nm. It thereby becomes possible to excite the detection pixels without bound and marked test substance to produce a photosignal, whereas no photoelectrons can be generated in the detection pixels with test substance as a result of the absorption dye.

There are then various possibilities for configuring and developing the teaching of the present invention in an advantageous manner. In this respect, reference should be made firstly to the patent claims subordinate to patent claim 1, and secondly to the following explanation of the preferred exemplary embodiments of the invention with reference to the drawing. Generally preferred configurations and developments of the teaching are also explained in conjunction with the explanation of the preferred exemplary embodiments of the invention with reference to the drawing. In the drawing, in each case in a schematic illustration,

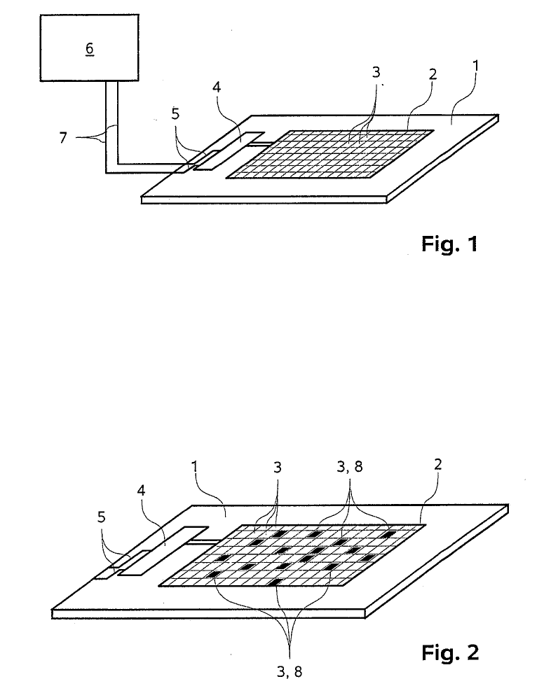

FIG. 1 shows, in a perspective view, an exemplary embodiment of a microchip connected to a control and read-out computer,

FIG. 2 shows the microchip from FIG. 1, on which connection objects are applied in a first method step,

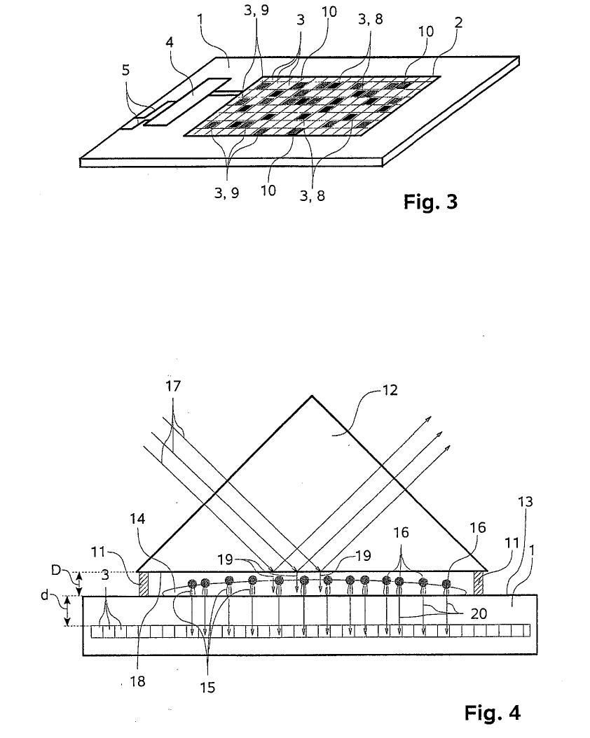

FIG. 3 shows the microchip from FIG. 2, on which further connection objects are applied in a further method step,

FIG. 4 shows, in a sectional view, a microchip on which objects are arranged which are illuminated evanescently,

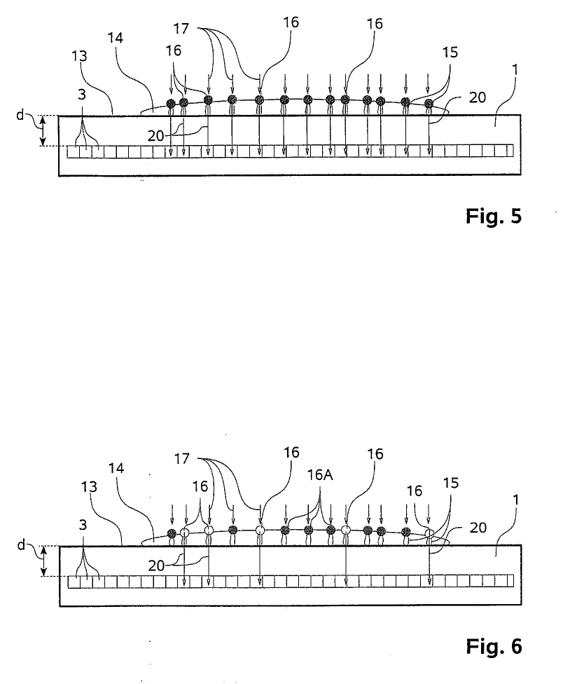

FIG. 5 shows, in a sectional view, a microchip on which objects are arranged which are illuminated areally for excitation of fluorescence,

FIG. 6 shows, in a sectional view, a microchip on which objects marked with an absorption dye are arranged, which objects are illuminated areally, and

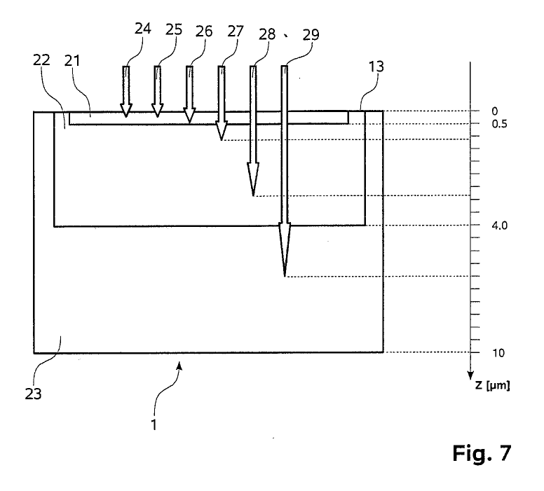

FIG. 7 shows, in a sectional view, a part of a microchip.

In the Figures, identical or similar components are identified by the same reference symbols.

FIG. 1 shows a microchip 1 on which objects (not shown in FIG. 1) can be applied and fixed at a respectively predetermined position. The microchip 1 has a region 2 in which detection pixels 3 are arranged. Light-sensitive electronic units embodied in the form of photodiodes are provided as detection pixels 3. The microchip 1, and in particular the region 2 thereof, is based on CMOS technology and is therefore an electronic semiconductor component. The microchip 1 is designed specifically for the detection of biological or chemical objects. The microchip 1 has—indicated only schematically—integrated electronic circuits 4 for driving or for reading the detection pixels 3 and the further elements of the microchip 1. The integrated electronic circuits 4 also serve for amplifying and conditioning the signals generated by the detection pixels 3. The microchip 1 comprises a driving and reading interface 5, which is likewise illustrated schematically just in the form of line connections. On the microchip 1, as it were, the interface is an I²C interface. The microchip 1 is connected via the external line connections 7 to a control computer 6, which drives the microchip 1 and by means of which the measured information or signals from the detection pixels 3 of the microchip 1 can be read out. Externally with respect to the control computer 6, the microchip 1 is connected via a USB interface.

In the region 2 of the microchip 1, for each detection pixel 3 in each case an electrode pixel is provided, which is not identified by its own reference symbol but is arranged nearer to the surface of the microchip 1 compared with the detection pixels 3. Accordingly, the detection pixels 3 are arranged at a further distance from the surface of the microchip 1.

FIG. 2 shows the microchip 1 from FIG. 1 in a state in which a first layer of connection objects 8 have been specifically applied to the microchip 1, or the region 2, and bound there. The connection objects 8 are depicted merely schematically as black quadrangles. The connection objects 8 were electrostatically negatively charged and applied to the microchip 1 from an aerosol (in comparison with the toner in a laser printer or according to EP 1 140 977 B1). A voltage was selectively applied to the corresponding electrode pixels (likewise identified hereinafter by the reference symbol 3 in FIGS. 1 to 3), with the result that a positive electric field formed, whereby the negatively charged connection object 8 situated in the aerosol became attached to the surface of the region 2 of the microchip 1 and thus in direct spatial proximity to the electrode pixels 3. Since the microchip 1, for electrical insulation with respect to the applied solution mixture, is coated with a silicon nitride layer (not shown in the Figures) and thereon in turn with a polyethylene glycol layer that promotes the attachment of connection objects 8, the connection objects 8 can be fixed to the respective surface. In FIG. 3, this method step was repeated, in which case other connection objects 9 (depicted in a hatched manner) were then applied in the region 2 of the microchip 1. In this case, for the most part other electrode pixels 3 were activated, with the result that at these locations a first layer of the connection objects 9 became attached on the surface of the microchip 1. At locations that are defined by the reference symbol 10, the corresponding electrode pixels 3 were also activated during this attachment process, with the result that a further layer of connection objects 9 became attached to the connection object 8 attached to this location. This method step can then be repeated a number of times, such that as a result a predetermineable sequence of different connection objects 8, 9 became specifically attached to the respective electrode pixels and detection pixels. The objects to be detected can then be specifically attached to these specific sequences of connection objects. A connection object can comprise a reactive molecule, for example an oligomer, a peptide oligomer, a DNA oligomer or a PNA oligomer of defined amino acid or nucleotide sequence. An object can comprise an antibody or an antibody mixture, proteins, peptides, DNA molecules, RNA molecules, PNA molecules, sugar molecules or bacterial lipopolysaccharide.

FIG. 4 shows a part of the microchip 1 in a sectional illustration. On the microchip 1, a microprism 12 is arranged at a predetermineable distance D from the surface 13 of the microchip 1 by positioning means 11. Likewise on the surface 13 of the microchip 1 there is schematically shown the insulation coating and the layer—with the reference symbol 14—which contains the synthesized connection objects—shown with the reference symbol 15 in FIGS. 4 to 6—and to which the objects 16 to be detected are specifically attached. The Figure likewise indicates merely schematically that illumination light 17 is coupled into the prism 12 in such a way that it is totally reflected internally at the surface 18 of the microprism 12 that faces the surface 13 of the microchip 1. The arrows 19 indicate the evanescent light field which forms on account of the total reflection of the illumination light 17 and which propagates in the direction of the surface 13 of the microchip 1. The intensity of the evanescent light 19 decreases exponentially with the distance from the surface 18 of the prism 12, such that the evanescent light 19 in any case illuminates the objects 16 and, if appropriate, also penetrates slightly into the microchip 1. The objects 16 are specifically marked with a fluorescent dye and emit fluorescence light 20 on account of the excitation with the evanescent light 19. The fluorescence light 20 propagates in all directions, but the arrows only indicate the portions which are detected by the detection pixels 3. The detection pixels 3 are arranged at a distance d from the surface 13 of the microchip 1. Since the fluorescence light 20 has a wavelength that is greater than the wavelength of the illumination light 17, the fluorescence light 20 also has a larger penetration depth into the microchip 1, such that the fluorescence light 20 can be detected by the detection pixels arranged at the distance d from the surface 13 of the microchip 1.

FIG. 5 likewise shows, in a sectional view, the microchip 1 from FIG. 4, on which objects 16 are likewise specifically attached. In the exemplary embodiment in accordance with FIG. 5, the objects 16 are illuminated essentially areally with illumination light 17. The objects 16 are specifically marked by means of nanocrystals (not depicted separately). In this exemplary embodiment, too, the nanocrystals are excited to fluorescence by means of the illumination light 17 having a wavelength of 280 nm. Since the illumination light 17 has a relatively short wavelength, its penetration depth into the microchip 1 is only very small, such that the illumination light 17 cannot in any case reach the detection pixels 3. By contrast, the fluorescence light from the nanocrystals has a sufficient penetration depth (greater than d), such that the fluorescence light 20 can be detected by the detection pixels 3. Although this is not illustrated in FIG. 5, the illumination light 17 could also be focused onto individual objects 16, such that the latter can be selectively excited. In this exemplary embodiment, it is also possible to excite the objects 16 to fluorescence by means of pulsed illumination light, in which case the detection pixels 3 are read only in the illumination pauses or their signals are taken into account only in the illumination pauses.

Essentially the same situation as shown in FIG. 5 is shown in the exemplary embodiment in accordance with FIG. 6. However, in this exemplary embodiment, some of the objects 16A shown in FIG. 5 are specifically marked with an absorption dye. These objects are depicted darker. The remaining objects 16 are not marked with the absorption dye. The objects 16, 16A or the microchip 1 is illuminated with illumination light 17. The arrows 20 indicate that the illumination light 17 can propagate through the objects 16 when the latter are not marked with the absorption dye. The corresponding detection pixels 3 to which the respective arrows 20 point can therefore detect the illumination light 17. However, the illumination light 17 cannot pass through the objects 16A specifically marked with absorption dye, with the result that the detection pixels 3 arranged underneath cannot detect a light signal.

FIG. 7 shows, in a sectional view, a part of the microchip 1 in which the detection pixels 3 (not shown explicitly in FIG. 7) are also arranged. Shown on the right alongside the microchip 1 is a scale showing the distance Z from the surface 13 of the microchip in μm. Various regions in the microchip 1 are furthermore shown. By way of example, the reference symbol 21 shows the PPLUS, 22 the NWELL and 23 the PEPI region of a photodiode. The arrows 24 to 29 are intended to represent the penetration depths of light having respectively different wavelengths.

In this case, the arrow 24 represents a wavelength of approximately 260 nm, the arrow 25 represents a wavelength of approximately 350 nm; the arrow 26 represents a wavelength of approximately 480 nm, the arrow 27 represents a wavelength of approximately 520 nm, the arrow 28 represents a wavelength of approximately 620 nm, and the arrow 29 represents a wavelength of approximately 750 nm. From the scale it is possible to read how deeply the light having the respective wavelength can penetrate into the microchip 1 into the silicon. Accordingly, through a suitable choice of the wavelength of the illumination light or given properties of the microchip 1 (e.g. for a given distance d of the detection pixels 3 from the surface 13 of the microchip), it is possible to choose a suitable fluorescent dye with which the objects 16 are then to be specifically marked, such that only the fluorescence light emerging from the objects (or that from the fluorescent dye bound to the objects) can penetrate into the microchip as far as the detection pixels 3, but the illumination or excitation light is unable to do this.

Finally, it should especially be pointed out that the exemplary embodiments discussed above serve only for describing the claimed teaching, but do not restrict said teaching to the exemplary embodiments.

1. A method for the detection of at least one property of at least one object, wherein the detection is effected by means of a microchip, wherein the microchip has at least one readable detection pixel,
wherein the at least one object is arranged at the microchip in a spatially predetermineable position, and wherein the at least one object is exposed to illumination light in order to detect the illumination light that interacts with the at least one object or the light that is induced by the illumination light and emerges from the at least one object by means of the at least one readable detection pixel of the microchip.

2. The method as claimed in claim 1, wherein the microchip has at least one electrode pixel, wherein an electrode pixel is embodied in the form of a high-voltage pixel or a high-voltage electrode, wherein a voltage lying in a range of 30 to 100 V is applied to an electrode pixel.

3. The method as claimed in claim 2, wherein the at least one object or at least one connection object is attached to the microchip in such a way that an electric field is generated selectively by means of the at least one electrode pixel, whereby at least one object or at least one connection object is attached to a surface region of the microchip which is assigned to the electrode pixel.

4. The method as claimed in claim 3, wherein the selective attachment of connecting objects is repeated, whereby a plurality of connection objects are synthesized pixel-by-pixel.

5. The method as claimed in claim 3, wherein at least one object is specifically attached to the at least one connection object, wherein the at least one object constitutes or comprises an antibody or an antibody mixture, proteins, peptides, DNA molecules, RNA molecules, PNA molecules, sugar molecules or bacterial lipopolysaccharide.

6. The method as claimed in claim 1, wherein at least one specific and/or synthesized connection object are applied and/or bound to the microchip surface, wherein the connection objects are embodied in such a way that the objects to be examined in each case specifically bind to them.

7. The method as claimed in claim 3, wherein a connection object comprises a reactive molecule, in particular an oligomer, a peptide oligomer, a DNA oligomer or a PNA oligomer of defined amino acid or nucleotide sequence.

8. The method as claimed in claim 1, wherein the at least one object is positioned by applying a film on the microchip, wherein the at least one object is positioned on the film in a spatially predetermineable manner or wherein the at least one object is at least partly surrounded by a medium, wherein the medium is embodied in such a way that the relative position of the objects remains essentially unchanged thereby, and wherein the medium together with the objects is applied to the microchip.

9. (canceled)

10. The method as claimed in claim 1, wherein the at least one object is illuminated in punctiform or areal fashion with illumination light having at least one predetermineable wavelength or a predetermineable wavelength range, wherein the wavelength range extends from 280 nm to 1000 nm.

11. The method as claimed in claim 1, wherein the at least one object is illuminated evanescently, or wherein the at least one object is illuminated evanescently with the aid of a prism which is arranged at a predetermineable distance relative to the microchip and into which illumination light is coupled in such a way that an evanescent field forms, with which the at least one object is illuminated.

12. The method as claimed in claim 1, wherein the illumination light is generated by means of a light source that emits continuous or pulsed light, and wherein the light source comprises a laser, a thermal radiator or a gas discharge lamp.

13. The method as claimed in claim 12, wherein the object is illuminated with pulsed light, and wherein the at least one detection pixel is read in an illumination pause.

14. The method as claimed in claim 1, wherein the object is marked with an absorption dye, and wherein that proportion of the illumination light which passes through the object to the respective detection pixel is detected.

15. The method as claimed in claim 1, wherein the object is specifically marked with at least one luminescent dye, or wherein the object is specifically marked with at least one nanocrystal capable of luminescence.

16. The method as claimed in claim 15, wherein the at least one luminescent dye is excited to luminescence by the illumination light, and wherein the luminescence light is detected by a detection pixel or wherein the luminescent dye comprises a fluorescent dye or a phosphorescent dye, or wherein the nanocrystal is capable of fluorescence or luminescence.

17. (canceled)

18. The method as claimed in claim 15, wherein the nanocrystal has a predetermineable hydrodynamic radius, or wherein the nanocrystal has a predetermineable excitation and emission spectrum, which has a high Stokes shift.

19. The method as claimed in claim 15, wherein the nanocrystal comprises a core comprising a semiconductor material or a lanthanide material, for example a europium compound, or wherein the nanocrystal comprises a coating that promotes a specific binding of the nanocrystal to an object.

20. The method as claimed in claim 16, wherein the fluorescent dye has a predetermineable high Stokes shift, and wherein the fluorescent dye could comprise lanthanide chelate.

21. The method as claimed in claim 16, wherein the fluorescent dye or the fluorescent nanocrystal has a predetermineable fluorescence lifetime, or wherein the fluorescent dye or the fluorescent nanocrystal has a predetermineable fluorescence lifetime being greater than or equal to 1 ms, wherein the objects are specifically marked with the fluorescent dye or the fluorescent nanocrystal, wherein the objects are illuminated with pulsed illumination light, and wherein the detection pixels are read in the illumination pauses.

22. The method as claimed in claim 1, wherein at least one detection pixel is provided which detects the illumination light, wherein on the basis of the detection signal of the detection pixel it is ascertained whether an illumination pause is present, or wherein on the basis of the detection signal of the detection pixel—for calibration, for example—it is possible to infer the local illumination situation, in particular the local illuminance.

23. The method as claimed in claim 1, wherein the objects are specifically marked with at least two fluorescent dyes having different excitation properties, wherein one of the fluorescent dyes is excited to fluorescence by means of illumination light having a first excitation wavelength for a predetermineable time interval, wherein afterward the other fluorescent dye is excited to fluorescence by means of illumination light having a second excitation wavelength for a further predetermineable time interval, and wherein the fluorescence light from the two fluorescent dyes is detected temporally successively.

24. The method as claimed in claim 1, wherein the objects are specifically marked with at least two fluorescent dyes having different emission properties, wherein the two fluorescent dyes are excited to fluorescence by means of illumination light having a predetermineable wavelength, wherein the fluorescence light from the first fluorescent dye has a first predetermineable penetration depth into the microchip, wherein the fluorescence light from the second fluorescent dye has a second predetermineable penetration depth into the microchip, wherein the first penetration depth is greater than the second penetration depth, and wherein the detection region of the detection pixels is arranged at least two different distances from the microchip surface, such that the fluorescence light from the first fluorescent dye is detected by the detection pixels that are at a further distance from the microchip surface and the fluorescence light from the second fluorescent dye is detected by the detection pixels that are at a lesser distance from the microchip surface.

25. The method as claimed in claim 1, wherein the at least one object is exposed to an electromagnetic wave instead of illumination light, in order to detect the electromagnetic wave that interacts with the at least one object or an electromagnetic wave that is induced by the electromagnetic wave and emerges from the at least one object by means of the at least one readable detection pixel of the microchip.

26. A device for the detection of at least one property of at least one object, in particular for carrying out a method as claimed in claim 1, comprising a microchip having at least one readable detection pixel,
wherein the at least one object is arranged at the microchip in a spatially predetermineable position, and wherein the at least one object is exposed to illumination light in order to detect the illumination light that interacts with the at least one object or the light that is induced by the illumination light and emerges from the at least one object by means of the at least one readable detection pixel of the microchip.

27. The device as claimed in claim 26, wherein the microchip is based on MOS technology, or on CMOS technology, on NMOS technology or on PMOS technology or wherein a detection pixel has a light-sensitive electronic unit or a photodiode or a photogate.

28. (canceled)

29. The device as claimed in claim 26, wherein the microchip has integrated electronic circuits for driving or for reading the detection pixels or the electrode pixels, and wherein the detection pixels is read individually or in groups.

30. The device as claimed in claim 26, wherein the microchip has at least one electrode pixel which is embodied in the form of a high- or low-voltage pixel or wherein the microchip has at least one driving or read-out interface which is embodied, in particular in the form of an I²C or USB interface.

31. (canceled)

32. The device as claimed in claim 26, wherein the microchip is driven or read by a control computer or wherein the microchip has means for amplifying or conditioning the signals that are read from a detection pixel.

33. (canceled)

34. The device as claimed in claim 26, wherein the microchip comprises a coating for electrical insulation, or wherein the microchip comprises a coating for electrical insulation, said coating comprising silicon nitride, or wherein a layer to which connection objects or objects are attached is applied on the microchip, and wherein the layer comprises at least one type of a polymer or an element of the type of the polyethylene glycols or a silanization layer, and wherein the layer comprises, in particular, a mixture of these substances.

35. (canceled)
